(19) United States
(12) Patent Application Publication
(10) Pub. No.: US 2023/0414209 A1
(43) Pub. Date: Dec. 28, 2023

(54) GASTROINTESTINAL SURGERY LIGHT-EMITTING TUBE CAPABLE OF SUCTION AND POSITION ADJUSTMENT

(71) Applicant: KOREA UNIVERSITY RESEARCH AND BUSINESS FOUNDATION, Seoul (KR)

(72) Inventor: Sungsoo PARK, Seoul (KR)

(73) Assignee: KOREA UNIVERSITY RESEARCH AND BUSINESS FOUNDATION, Seoul (KR)

(21) Appl. No.: 18/039,721

(22) PCT Filed: Jul. 6, 2021
(86) PCT No.: PCT/KR2021/008578

§ 371 Date: Jul. 3, 2023

(30) Foreign Application Priority Data
Dec. 3, 2020 (KR) 10-2020-0167326

(51) Int. Cl. A61B 17/00

(52) U.S. Cl. CPC A61B 17/00234; A61B 2217/005; A61B 2017/00818; A61B 2017/003

(57) ABSTRACT

The present disclosure relates to a gastrointestinal surgery light-emitting tube capable of suction and position adjustment including a body that has a proximal end and a distal end, is a hollow having an outer surface and an inner surface, and opens at the proximal end; and a shape-changing balloon that is located at the distal end of the body, in which in a cross section of the shape-changing balloon in an inflated state, a length of a portion where the shape-changing balloon comes into contact with the body is longer than a length of a portion where the shape-changing balloon does not come into contact with the body.

### TECHNICAL FIELD

The present disclosure relates to a gastrointestinal surgery light-emitting tube capable of suction and position adjustment, and more specifically, to a gastrointestinal surgery light-emitting tube capable of suction and position adjustment that is capable of performing suction and position control, and easy to locate a position using light.

### BACKGROUND

This section provides background information related to the present disclosure which is not necessarily prior art.

One example of gastrointestinal surgery is sleeve gastrectomy performed for the treatment of obese patients.

In general, a body mass index is obtained by dividing the body weight by the square of the height (kg/m2) and if the value of this body mass index is 30 or more, Asians are said to be the sever obesity.

In the case of Asians, the final diagnosis is made by examining the visceral fat percentage, abdominal fat percentage, and body fat percentage in detail along with the simple weight.

In this way, when the patient becomes the sever obesity, the patient's health is seriously adversely affected, and the risk of developing diabetes, hypertension, hyperlipidemia, fatty liver, etc., which are representative complications of obesity, increases rapidly.

A case where highly obese patients have a history of failure in conservative treatment methods such as diet, exercise, behavior, and habit correction, a case where their body mass index is 30 or more and diabetes, hypertension, hypercholesterolemia, fatty liver, and arthritis, sleep apnea, etc. caused by obesity are accompanied, a case where the body mass index is 35 or more that is ultra-obesity, and a case of severe morbid abdominal obesity which is a characteristic form of obesity in Asians are also classified as severe obesity, and surgical treatment is considered.

As a surgical method, sleeve gastrectomy is used. Sleeve gastrectomy is a method of leaving the antrum (lower part) of the stomach, which has an important digestive function in the stomach, and reducing the large portion of the stomach along the curve of the small portion to make the stomach tube-shaped. In sleeve gastrectomy, an operator uses a tube such as a gastric tube to assist in the procedure.

FIG. 1 is a view showing an example of a mouth catheter for longitudinal gastric ablation described in Korean Patent Registration No. 10-1906411. For convenience of description, some drawing symbols and names have been changed.

A mouth catheter 10 includes a hollow body 11 having a proximal end 12 and a distal end 13 and a balloon 14 located at the distal end 13, and the body 11 includes a hole 15 through which fluid (eg, gastric juice, gas, etc.) passes. The sleeve gastrectomy may be easily performed by the operator by using the balloon 14 to accurately position the mouth catheter 10.

However, in the mouth catheter 10 of the prior art described in FIG. 1, the balloon 14 is configured such that the distal end of the balloon 14 comes into contact with the patient's pylorus in order to accurately position the mouth catheter 10. Accordingly, the distal end 13 of the mouth catheter 10 has a structure farther away from the patient's pylorus than the balloon. As a result, it is difficult for the operator to make the shape of the mouth catheter 10 into an appropriate shape (eg, “J” shape) for sleeve gastrectomy.

In the present disclosure, the operator may easily make, as a “J” shape, a shape of a gastrointestinal surgery light-emitting tube capable of suction and position adjustment in the state of being inserted into the stomach, and furthermore, a gastrointestinal surgery light-emitting tube capable of suction and position adjustment is provided in which the position of the gastrointestinal surgery light-emitting tube capable of suction and position adjustment control may be easily recognized by the operator. Provides a light-emitting tube for gastrointestinal surgery that and positioned.

### SUMMARY OF INVENTION

### Technical Problem

This will be described at the end of ‘BEST MODE FOR INVENTION’.

### Technical Solutions

This section provides a general summary of the disclosure and is not a comprehensive disclosure of its full scope or all of its features

According to one aspect of the present disclosure, there is provided a gastrointestinal surgery light-emitting tube capable of suction and position adjustment including a body that has a proximal end and a distal end, is a hollow having an outer surface and an inner surface, and opens at the proximal end; and a shape-changing balloon that is located at the distal end of the body, in which in a cross section of the shape-changing balloon in an inflated state, a length of a portion where the shape-changing balloon comes into contact with the body is longer than a length of a portion where the shape-changing balloon does not come into contact with the body.

### Advantageous Effects

This will be described at the end of ‘BEST MODE FOR INVENTION’.

### BRIEF DESCRIPTION OF THE DRAWINGS

FIG. 1 is a view showing an example of a mouth catheter for longitudinal gastric ablation described in Korean Patent Registration No. 10-1906411,

FIG. 2 is a view showing an example of a gastrointestinal surgery light-emitting tube capable of suction and position adjustment according to the present disclosure,

FIG. 3 is a view showing another example of the gastrointestinal surgery light-emitting tube capable of suction and position adjustment according to the present disclosure,

FIG. 4 is a view showing another example of the gastrointestinal surgery light-emitting tube capable of suction and position adjustment according to the present disclosure,

FIG. 5 is a view showing an example of use of the gastrointestinal surgery light-emitting tube capable of suction and position adjustment according to the present disclosure, and

FIG. 6 is a view showing another example of the gastrointestinal surgery light-emitting tube capable of suction and position adjustment according to the present disclosure.

### DESCRIPTION OF REFERENCE NUMBER

Gastrointestinal surgery light-emitting tube capable of suction and position adjustment: 10, 100
Body: 11, 110
Proximal end of body: 12, 111
Distal end of body: 13, 112
wire: 120
Balloons: 14, 130, 150
Light way: 140

### BEST MODE FOR INVENTION

The present disclosure will now be described in detail with reference to the accompanying drawings. In addition, direction indications such as upper/lower, up/down, etc. in this specification are based on drawings.

In the embodiments to be described later, a case where a gastrointestinal surgery light-emitting tube capable of suction and position adjustment according to the present disclosure is used for sleeve gastrectomy will be described, but the use of the gastrointestinal surgery light-emitting tube capable of suction and position adjustment is not necessarily limited thereto, and may be used in various types of gastrointestinal surgery.

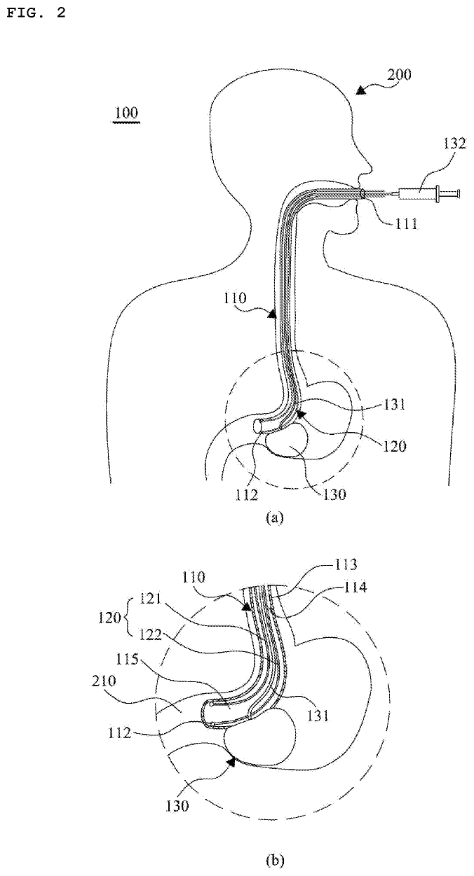

FIG. 2 is a view showing an example of the gastrointestinal surgery light-emitting tube capable of suction and position adjustment according to the present disclosure.

FIG. 2(a) shows a shape in which the gastrointestinal surgery light-emitting tube capable of suction and position adjustment is used on a patient, and FIG. 2(b) is an enlarged view of a dotted circle and is a sectional view showing the gastrointestinal surgery light-emitting tube capable of suction and position adjustment for description.

A gastrointestinal surgery light-emitting tube capable of suction and position adjustment 100 may include a body 110 having a proximal end 111 and a distal end 112, a pair of wires 120 fixed to the distal end 112 of the body 110, and a volumetric balloon 130 located at the distal end 112 of the body 110.

The body 110 may be formed of a material having elasticity (eg, silicon). In addition, the body 110 has a hollow 115 having an outer surface 113 and an inner surface 114, and the proximal end 111 is open. The proximal end 111 may be exposed to the outside of the patient when the gastrointestinal surgery light-emitting tube capable of suction and position adjustment 100 is inserted through the esophagus of the patient 200 to the stomach, and the operator may manipulate the wire 120 exposed through opened the proximal end 111.

The pair of wires 120 fixed to the distal end 112 of the body 110 are not excluded from being fixed to the outer surface 113 of the body 110, but is preferably fixed to the inner surface 114 of the body 110 in order to protect the patient's organs from the wires 120. Furthermore, it is preferable that the pair of wires 120 are located facing each other. The operator may adjust the direction of the distal end 112 of the body 110 by pulling the wire 120 exposed to the proximal end 111 of the body 110. For example, when the operator pulls a first wire 121, the distal end 112 may face left and when the operator pulls a second wire 122, the distal end 112 may face right. That is, the operator properly adjusts the first wire 121 and the second wire 122 to make the shape of the body 110 as “J” as shown in FIG. 2(b), and the distal end 112 of the body 110 may face the pylorus 210 of the patient 200.

The volumetric balloon 130 may be inflated by at least one of physiological saline and air supplied through a fluid supply pipe 131. For example, the volumetric balloon 130 may be inflated by supplying physiological saline using the syringe 132. The size of the volumetric balloon 130 may be adjusted by adjusting the amount of physiological saline solution which is supplied. The fluid supply pipe 131 is a pipe connected from the proximal end 111 of the body 110 to the volumetric balloon 130. The volumetric balloon 130 is preferably located farther from the pylorus 210 of the patient 200 than the distal end 112 of the body 110 in the inflated state. In the present disclosure, the volumetric balloon 130 is configured such that the body 110 has the “J” shape toward the pylorus 210 of the patient, and the operator accurately measures the volume of the antrum of the stomach to the extent desired by the operator without changing the J″ shape and then performs gastrectomy. In other words, during sleeve gastrectomy, it is a principle that the antrum of the stomach is maintained 4 to 6 cm from the pylorus 210 which is the boundary between the stomach and the duodenum, and the surgery is performed, but it is difficult to measure the volume of the anterior of the stomach, which is actually remained, due to various sizes and shapes of the antrum of the stomach for each patent. In the present disclosure, the volumetric balloon 130 may be used to accurately measure the volume of the antrum of the stomach to a degree desired by the operator, and then the “J” shape of the body 110 is unlikely to be deformed while operating gastrectomy.

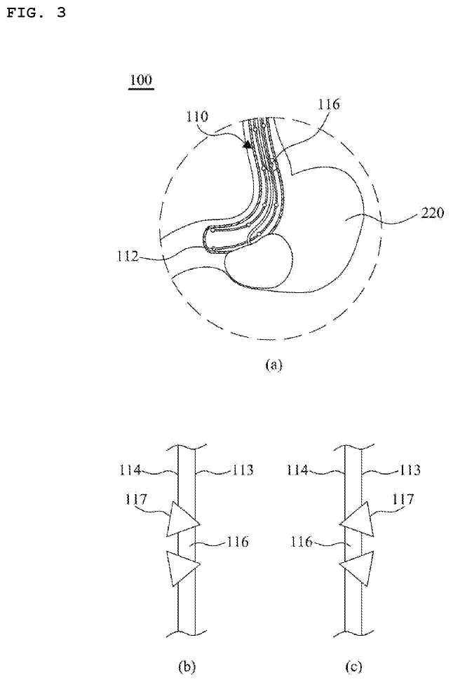

FIG. 3 is a view showing another example of the gastrointestinal surgery light-emitting tube capable of suction and position adjustment according to the present disclosure.

FIG. 3(a) is a perspective view and FIGS. 3(b) and 3(c) are enlarged sectional views of the hole 116 for explanation.

The gastrointestinal surgery light-emitting tube capable of suction and position adjustment 100 may include a plurality of holes 116 penetrating from the inner surface 114 to the outer surface 113 of the body 110 located on the patient's stomach 220 in the body 110. A suction function through the plurality of holes 116 avoids the volume change due to gas or gastric contents, and a washing function makes it easy to perform a leakage test after washing and resection, thereby preventing complications after the surgery. Furthermore, the body 110 may include a valve 117 that is opened only when a fluid flows through the plurality of holes 116 so that the fluid flows through the plurality of holes 116 only when necessary. For example, the valve 117 is formed of a softer material than the body 110 as shown in FIG. 3 (b), and in a case where a suction force is generated from the outside through the hollow of the gastrointestinal surgery light-emitting tube capable of suction and position adjustment 100 based on the valve 117, the suction force does not distort the shape of the body 110, but the valve 117 may be opened in the direction of the inner surface 114 of the body 110 by the suction force, and as shown in FIG. 3 (c), in a case where washing water is supplied from the outside through the hollow of the gastrointestinal surgery light-emitting tube capable of suction and position adjustment 100, the valve 117 may be opened in the direction of the outer surface 113 of the body 110 by the washing water. Except for those described in FIG. 3, the gastrointestinal surgery light-emitting tube capable of suction and position adjustment 100 is substantially the same as the gastrointestinal surgery light-emitting tube capable of suction and position adjustment 100 described in FIG. 2.

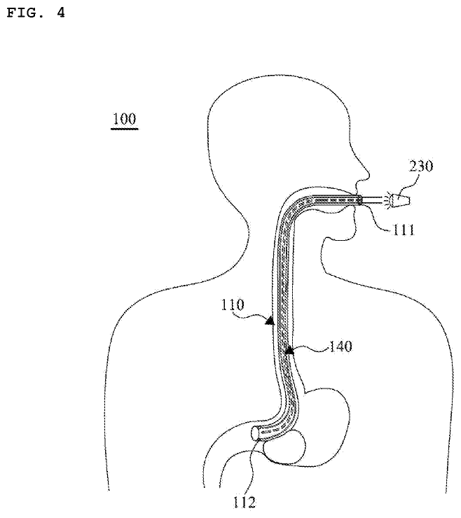

FIG. 4 is a view showing another example of the gastrointestinal surgery light-emitting tube capable of suction and position adjustment according to the present disclosure.

The gastrointestinal surgery light-emitting tube capable of suction and position adjustment 100 may include a plurality of light ways 140 on at least one of the outer surface 113 and the inner surface 114 of the body 110 from the proximal end 111 to the distal end 112 of the body 110. Light emitted from the light way 140 transmits through the gastric wall, so that the operator may recognize the gastrointestinal surgery light-emitting tube capable of suction and position adjustment 100 in real time, and thereby accurate surgery may be performed. The light way 140 may be formed of an optical fiber or an LED. When the light way 140 is formed of the optical fiber, light emitted from the external light source 230 located at the proximal end 111 of the body 110 may be used. In this case, since the external light source 230 may use various conventional light sources used during surgery, it is preferable that a separate light source for the gastrointestinal surgery light-emitting tube capable of suction and position adjustment 100 is not required. In addition, when the light way 140 is located on the inner surface of the body 110, it is preferable that the body 110 is formed of a translucent material so that the light emitted from the light way 140 may pass through the body 110. Except for those described in FIG. 4, the gastrointestinal surgery light-emitting tube capable of suction and position adjustment 100 is substantially the same as the gastrointestinal surgery light-emitting tube capable of suction and position adjustment 100 described in FIG. 2.

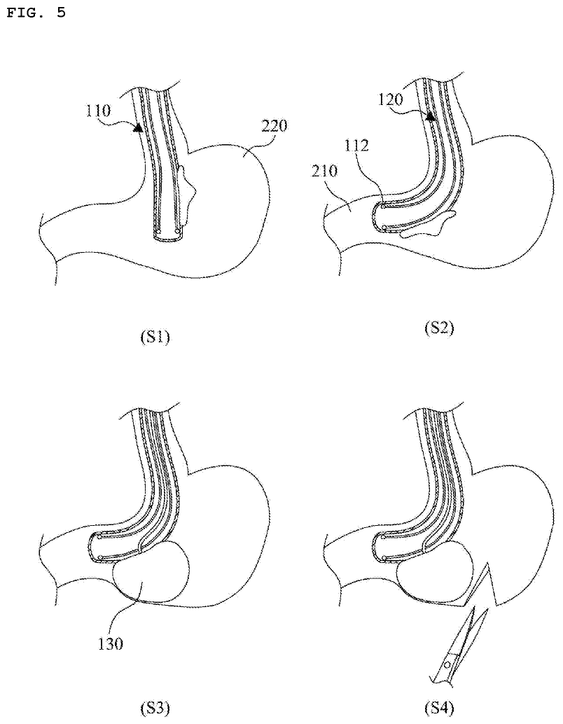

FIG. 5 is a view showing an example of use of the gastrointestinal surgery light-emitting tube capable of suction and position adjustment according to the present disclosure.

The body 110 of the gastrointestinal surgery light-emitting tube capable of suction and position adjustment 100 is inserted into the stomach 220 through the patient's esophagus (not shown) (S1). Then, using the wire 120 of the gastrointestinal surgery light-emitting tube capable of suction and position adjustment 100, the direction the distal end 112 faces the patient's pylorus 210 to make a “J” shape (S2). Thereafter, the volumetric balloon 130 is inflated to accurately measure the volume of the antrum of the stomach to the extent desired by the operator (S3). Then, gastrectomy is performed (S4).

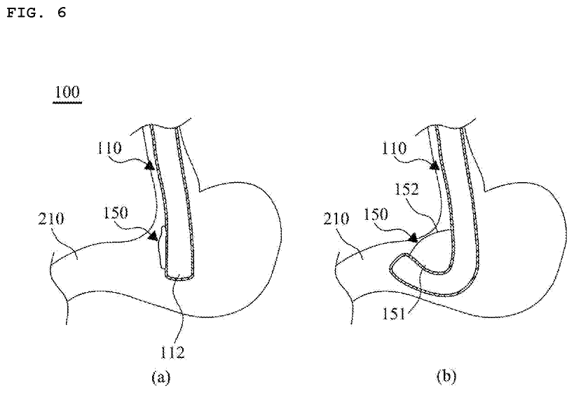

FIG. 6 is a view showing another example of the gastrointestinal surgery light-emitting tube capable of suction and position adjustment according to the present disclosure.

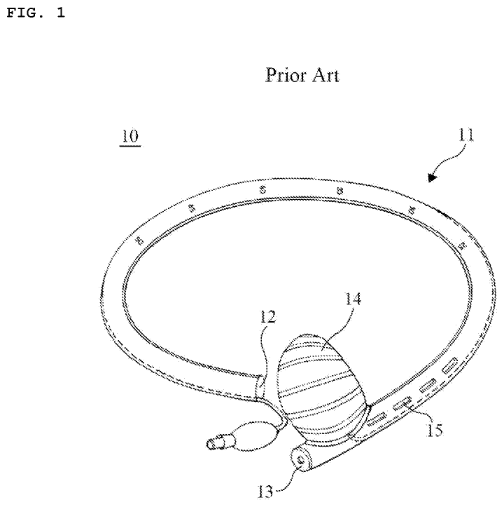

The gastrointestinal surgery light-emitting tube capable of suction and position adjustment 100 may include a shape-changing balloon 150 so that the direction of the distal end 112 faces the patient's pylorus 210 and thereby the body 110 of the gastrointestinal surgery light-emitting tube capable of suction and position adjustment 100 has the “J” shape. That is, the conventional balloon 14 described in FIG. 1 is for fixing the position, and the volumetric balloon 130 described in FIG. 3 enables the operator to accurately measure the volume of the antrum of the stomach to a desired degree, and the shape of the body 110 of the gastrointestinal surgery light-emitting tube capable of suction and position adjustment 100 is not changed. However, the shape-changing balloon 150 may make the shape of the gastrointestinal surgery light-emitting tube capable of suction and position adjustment 100 the “J” shape while being inflated. A method of inflating the shape-changing balloon 150 is substantially the same as that of the volumetric balloon 130 described in FIG. 3. In order for the shape-changing balloon 150 to make the shape of the body 110 the “J” shape, the shape-changing balloon 150 is preferably similar to a triangular shape as shown in FIG. 6(b) when being inflated. For example, looking at FIG. 6(b) showing a cross section when the shape-changing balloon 150 is inflated, a length 151 of a portion where the shape-changing balloon 150 comes into contact with the body 110 is preferably longer than a length 152 of a portion where the shape-changing balloon 150 does not come into contact with the body 110. In addition, although not shown, it is preferable to have a mark at the proximal end of the body 110 to distinguish the left and right sides of the body 110 or a mark to know the direction in which the shape-changing balloon 150 is located. This is because the operator may easily know the direction of the shape-changing balloon 150 from the outside of the patient. Except for those described in FIG. 6, the gastrointestinal surgery light-emitting tube capable of suction and position adjustment 100 is substantially the same as the gastrointestinal surgery light-emitting tube capable of suction and position adjustment described in FIG. 2. However, the wire 120 and the volumetric balloon 130 described in FIG. 2 may be included only when necessary. For example, if the “J” shape of the body 110 is not well formed only with the shape-changing balloon 150, the “J” shape of the body 110 may be better formed by adding the wire 120.

Hereinafter, various embodiments of the present disclosure will be described.
(1) A gastrointestinal surgery light-emitting tube capable of suction and position adjustment including a body that has a proximal end and a distal end, is a hollow having an outer surface and an inner surface, and opens at the proximal end; and a shape-changing balloon that is located at the distal end of the body, in which in a cross section of the shape-changing balloon in an inflated state, a length of a portion where the shape-changing balloon comes into contact with the body is longer than a length of a portion where the shape-changing balloon does not come into contact with the body.
(2) The gastrointestinal surgery light-emitting tube capable of suction and position adjustment in which a mark is provided at the proximal end of the body to indicate a direction in which the shape-changing balloon is located.
(3) The gastrointestinal surgery light-emitting tube capable of suction and position adjustment further including a volumetric balloon that is located at the distal end of the body, in which in a state where the volumetric balloon is inflated, the inflated volumetric balloon is located farther from the patient's pylorus than the distal end of the body.
(4) The gastrointestinal surgery light-emitting tube capable of suction and position adjustment further including a pair of wires fixed to the distal end of the body in which the pair of wires face each other on the inner surface of the body.
(5) The gastrointestinal surgery light-emitting tube capable of suction and position adjustment further including a fluid supply pipe that is connected from the proximal end of the body to the shape-changing balloon, and is a passage through which at least one of physiological saline and air is supplied to the shape-changing balloon.
(6) The gastrointestinal surgery light-emitting tube capable of suction and position adjustment in which the body includes a plurality of holes penetrating from the inner surface to the outer surface of the body.
(7) The gastrointestinal surgery light-emitting tube capable of suction and position adjustment further including a valve that is opened only when a fluid flows through a plurality of holes.
(8) The gastrointestinal surgery light-emitting tube capable of suction and position adjustment further including a plurality of light ways formed on at least one of the outer surface and the inner surface of the body from the proximal end to the distal end of the body.
(9) The gastrointestinal surgery light-emitting tube capable of suction and position adjustment in which the light way is formed of optical fiber.
(10) The gastrointestinal surgery light-emitting tube capable of suction and position adjustment in which the body is formed of a light transmitting material so that light emitted from the light way is capable of passing through the body when the light way is formed on the inner surface of the body.

According to the present disclosure, it is possible to obtain a gastric tube for sleeve gastrectomy in which the position may be adjusted and the position may be identified.

1. A gastrointestinal surgery light-emitting tube capable of suction and position adjustment comprising:
a body that has a proximal end and a distal end, is a hollow having an outer surface and an inner surface, and opens at the proximal end; and
a shape-changing balloon that is located at the distal end of the body,
wherein in a cross section of the shape-changing balloon in an inflated state, a length of a portion where the shape-changing balloon comes into contact with the body is longer than a length of a portion where the shape-changing balloon does not come into contact with the body.

2. The gastrointestinal surgery light-emitting tube capable of suction and position adjustment of claim 1, wherein a mark is provided at the proximal end of the body to indicate a direction in which the shape-changing balloon is located.

3. The gastrointestinal surgery light-emitting tube capable of suction and position adjustment of claim 1, further comprising:
a volumetric balloon that is located at the distal end of the body,
wherein in a state where the volumetric balloon is inflated, the inflated volumetric balloon is located farther from the patient's pylorus than the distal end of the body.

4. The gastrointestinal surgery light-emitting tube capable of suction and position adjustment of claim 1, further comprising:
a pair of wires fixed to the distal end of the body,
wherein the pair of wires face each other on the inner surface of the body.

5. The gastrointestinal surgery light-emitting tube capable of suction and position adjustment of claim 1, further comprising:
a fluid supply pipe that is connected from the proximal end of the body to the shape-changing balloon, and is a passage through which at least one of physiological saline and air is supplied to the shape-changing balloon.

6. The gastrointestinal surgery light-emitting tube capable of suction and position adjustment of claim 1, wherein the body includes a plurality of holes penetrating from the inner surface to the outer surface of the body.

7. The gastrointestinal surgery light-emitting tube capable of suction and position adjustment of claim 6, further comprising:
Wherein the body includes a valve that is opened only when a fluid flows through a plurality of holes.

8. The gastrointestinal surgery light-emitting tube capable of suction and position adjustment of claim 1, further comprising:
a plurality of light ways formed on at least one of the outer surface and the inner surface of the body from the proximal end to the distal end of the body.

9. The gastrointestinal surgery light-emitting tube capable of suction and position adjustment of claim 8, wherein the light way is formed of optical fiber.

10. The gastrointestinal surgery light-emitting tube capable of suction and position adjustment of claim 8, wherein the body is formed of a light transmitting material so that light emitted from the light way is capable of passing through the body when the light way is formed on the inner surface of the body.
